(19) United States
(12) Patent Application Publication
(10) Pub. No.: US 2008/0228164 A1
(43) Pub. Date: Sep. 18, 2008

(54) Implant delivery system

(76) Inventors: Zachary R. Nicoson, Indianapolis, IN (US); Aaron P. Barr, Fishers, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/717,825

(22) Filed: Mar. 14, 2007

(51) Int. Cl. A61M 25/00

(52) U.S. Cl. 604/506

(57) ABSTRACT

An embodiment includes a site marker deployment system that includes a deployment device having an inner cannula and a site marker that may be selectively interposed within the inner cannula. The site marker includes at least one generally elongated filament member. The filament member is selectively configurable between a first configuration, where the site marker can not be interposed within the inner cannula, and a second configuration, where the site marker may be interposed within the inner cannula. A portion of the site marker is elastically deformed when the site marker is interposed within the inner cannula. The site marker deployment system also includes a restraining member selectively interposed within the inner cannula and selectively engaging the site marker such that a force exerted on the restraining member may urge the site marker at least partially into the inner cannula.

### TECHNICAL FIELD

The present disclosure relates generally to site markers for breast biopsy procedures.

### BACKGROUND

In the diagnosis and treatment of breast cancer, it is often necessary to perform a biopsy to remove tissue samples from a suspicious mass. The suspicious mass is typically discovered during a preliminary examination involving visual examination, palpation, X-ray, magnetic resonance imaging (MRI), ultrasound imaging or other detection means.

When a suspicious mass is detected, a sample is taken by biopsy, and then tested to determine whether the mass is malignant or benign. This biopsy procedure can be performed by an open surgical technique, or through the use of a specialized biopsy instrument. To minimize surgical intrusion, a small specialized instrument such as a biopsy needle is inserted in the breast while the position of the needle is monitored using fluoroscopy, ultrasonic imaging, X-rays, MRI or other suitable imaging techniques.

In stereotactic needle biopsy, the patient lies on a special biopsy table with her breast compressed between the plates of a mammography apparatus and two separate X-rays are taken from two different points of reference. A computer then calculates the exact position of the mass or lesion within the breast. The coordinates of the lesion are then programmed into a mechanical stereotactic apparatus which advances the biopsy needle into the lesion with precision. At least five biopsy samples are usually taken from locations around the lesion and one from the center of the lesion.

Regardless of the method or instrument used to perform the biopsy, subsequent examination of the surgical site may be necessary, either in a follow up examination or for treatment of a cancerous lesion. Treatment often includes a mastectomy, lumpectomy, radiation therapy, or chemotherapy procedure that requires the surgeon or radiologist to direct surgical or radiation treatment to the precise location of the lesion. Because this treatment might extend over days or weeks after the biopsy procedure, and the original features of the tissue may have been removed or altered by the biopsy, it is desirable to insert a site marker into the surgical cavity to serve as a landmark for future identification of the location of the lesion.

However, some biopsy site markers may not be visible under all available modalities. When cancer is found at a biopsy site that has been previously marked with a site marker, the poor visibility of the biopsy site marker under ultrasound or other visualization modalities, may require that the patient undergo an additional procedure that places an additional device at the biopsy site to enable the surgeon to find the biopsy site in subsequent procedures. One known technique has been to place a breast lesion localization wire at the biopsy site. The localization wire is typically placed at the biopsy site via mammography and/or ultrasound.

Commonly assigned US Patent Publication 2006/0173296 discloses markers that use expandable filament portions to ‘hold’ a site marker in place within a biopsy cavity. That is, a site marker may include a bio-absorbable filament portion, such as a suture, with a marker attached thereto, where the marker is visible under multiple modalities and the suture will inhibit migration of the marker within the biopsy cavity. The filament portions of these structures typically define a site marker diameter that is greater than the outer diameter of the cannula. To insert a site marker within a biopsy site, the site marker is compressed (at least partially elastically deformed) to a dimension that will permit the site marker to be interposed within the cannula, the site marker is interposed within an opening of the cannula, the site marker and cannula are sterilized, the cannula is inserted within the biopsy canal such that the opening is within the biopsy site, and the marker is deployed into the biopsy site. Once deployed, the site marker will expand as the filament portions exit the cannula in reaction to the elastic deformation. The site marker will expand until the elastic deformation is eliminated or portions of the site marker interfere with the inside portions of the biopsy cavity.

The site marker and cannula must be sterile in order to be placed into a biopsy cavity. However, the elastically deformed filament portions, or other materials, plastically deform within the cannula due to the heat of sterilization (essentially converting some of the elastic deformation to plastic deformation) and may expand less upon exiting the cannula than a site marker that has no plastic deformation due to the heat of sterilization. Such a site marker may undesirably migrate within the biopsy cavity.

Accordingly, there is a need for site markers made from biocompatible materials that are visible under various modes of imaging to reduce the number of procedures that patients must undergo in detection and treatment of cancer, while being compatible with sterilization and packaging techniques.

### SUMMARY OF THE INVENTION

An embodiment includes a site marker deployment system that includes a deployment device having an inner cannula and a site marker that may be selectively interposed within the inner cannula. The site marker includes at least one generally elongated filament member. The filament member is selectively configurable between a first configuration, where the site marker can not be interposed within the inner cannula, and a second configuration, where the site marker may be interposed within the inner cannula. A portion of the site marker is elastically deformed when the site marker is interposed within the inner cannula. The site marker deployment system also includes a restraining member selectively interposed within the inner cannula and selectively engaging the site marker such that a force exerted on the restraining member may urge the site marker at least partially into the inner cannula.

### BRIEF DESCRIPTION OF THE DRAWINGS

Referring now to the drawings, illustrative embodiments are shown in detail. Although the drawings represent some embodiments, the drawings are not necessarily to scale and certain features may be exaggerated, removed, or partially sectioned to better illustrate and explain the present invention. Further, the embodiments set forth herein are not intended to be exhaustive or otherwise limit or restrict the claims to the precise forms and configurations shown in the drawings and disclosed in the following detailed description.

FIG. 1 is a perspective view of a biopsy site in a human breast showing the breast tissue in section and one or more site markers being implanted in the biopsy cavity using a site marker delivery system according to an embodiment.

FIG. 2 is an exploded side view of a site marker deployment system according to an embodiment.

FIG. 3 is a partially sectioned side view of a site marker and a portion of a deployment device.

FIG. 4 is a partially sectioned side view of the site marker and the deployment device of FIG. 3.

FIG. 5 is a partially sectioned side view of the site marker and the deployment device of FIG. 3.

FIG. 6 is a partially sectioned side view of the site marker and the deployment device of FIG. 3.

FIG. 7 is a partially sectioned side view of the site marker and the deployment device of FIG. 3 with additional components.

FIG. 8 is a partially sectioned side view of an alternative embodiment of a site marker deployment system.

FIG. 9 is a partially sectioned side view of the site marker deployment system of FIG. 8.

FIG. 9A is an enlarged portion of area 9A of FIG. 9.

FIG. 10 is a partially sectioned side view of the site marker and the deployment device of FIG. 8.

FIG. 11 is a partially sectioned side view of the site marker and the deployment device of FIG. 8.

FIG. 12 is a perspective view of a portion of the site marker and the deployment device of FIG. 8.

FIG. 13 is a partially sectioned side view of an alternative embodiment of a site marker deployment system.

FIG. 14 is a side view of a portion of the of a site marker deployment system of FIG. 13.

FIG. 15 is a side view of a portion of the of a site marker deployment system of FIG. 13.

FIG. 16 is a side view of a site marker within a cannula in accordance with another embodiment.

FIG. 17 is a side view of the marker of FIG. 16 in a deployed configuration.

### DETAILED DESCRIPTION

Referring now to the drawings, preferred illustrative embodiments are shown in detail. Although the drawings represent some embodiments, the drawings are not necessarily to scale and certain features may be exaggerated, removed, or partially sectioned to better illustrate and explain the present invention. Further, the embodiments set forth herein are not intended to be exhaustive or otherwise limit or restrict the claims to the precise forms and configurations shown in the drawings and disclosed in the following detailed description.

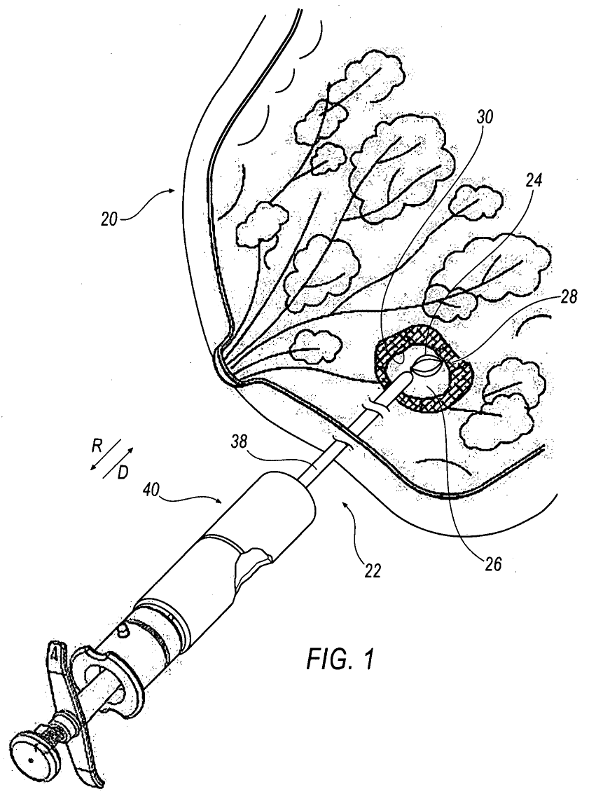

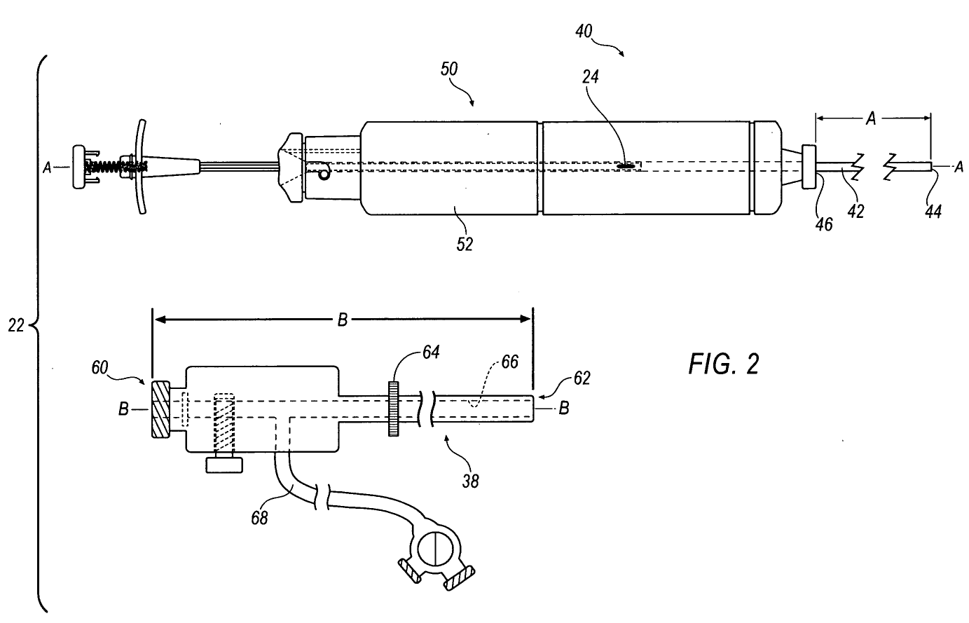

FIG. 1 illustrates a perspective view of a human breast, or tissue, 20 and a site marker deployment system 22. As illustrated, the tissue 20 is being implanted with one or more site markers 24 at a biopsy site 26. In the embodiment illustrated, the biopsy site 26 is a lesion 28 from which a tissue sample (not shown) has been removed, resulting in a biopsy cavity 30. One or more site markers 24 are implanted in the biopsy cavity 30 using the system 22. In one embodiment, the site marker delivery system 22 is slidably advanced through an outer cannula 38 (FIG. 2), which avoids the need to withdraw the biopsy device and thereafter insert the marker delivery system 22. The outer cannula 38 may be an introducer as illustrated in FIG. 2 that permits a biopsy device (not shown) to be inserted therein, or the outer cannula 38 may be a portion of the biopsy device. Delivering the site marker 24 in the biopsy cavity 30 without withdrawing the biopsy device reduces the amount of tissue damage and enables more accurate placement of the site marker 24. The system 22 illustrated in FIG. 1 is exemplary only and it is understood that the site marker embodiments disclosed herein are suitable for use with other marker delivery systems.

Referring to FIG. 2, the system 22 also includes an outer cannula 38 and a deployment device 40. The deployment device 40 includes an inner cannula 42 having a length A and an axis A-A and extending from a distal inner cannula end 44 to a proximal inner cannula end 46. The deployment device 40 also includes a body 50 having a handle 52.

The outer cannula 38 has an axis B-B and extends from an open proximal end 60 to an open distal end 62, which is separated from proximal end 60 by a distance B. The outer cannula 38 may be made from a medical grade resin or other MRI compatible material. A depth limiting member 64, such as a rubber o-ring, may be moveably disposed on outer cannula 38 to limit the insertion depth of outer cannula 38 into the patient's body. The outer cannula 38 also includes an inner lumen 66 therethrough, which is open to communication with a fluid conduit 68 for supplying fluids, such as saline and anesthetics, or removing fluids, such as blood, from the patient's body. In the embodiments illustrated, the distance A is slightly greater than the distance B.

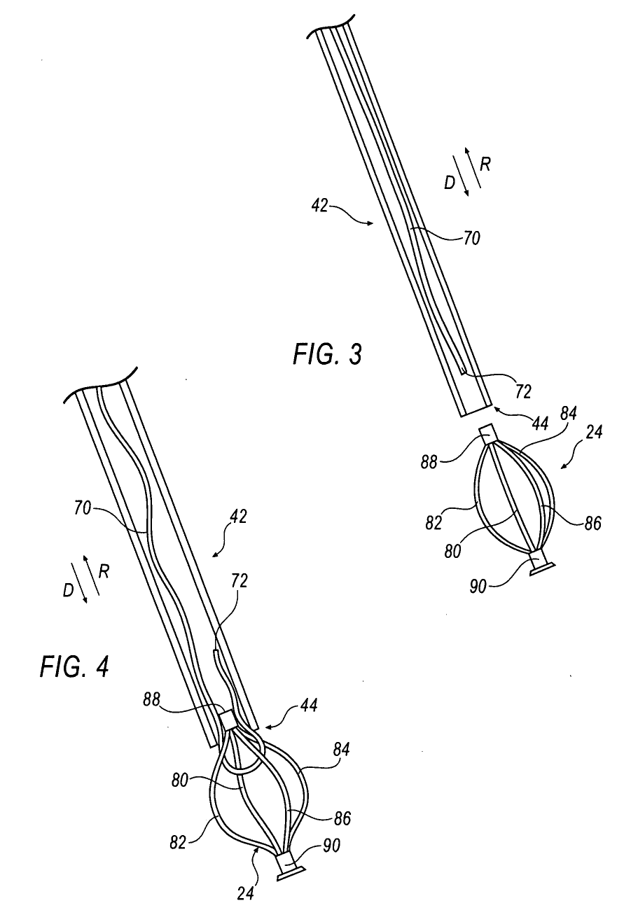

In the embodiment of FIGS. 3-7, the deployment device 40 includes a restraining member, or trigger wire, 70 interposed therein. The trigger wire 70 extends from a distal wire end 72 to a proximal wire end (not shown). As discussed in greater detail below, the trigger wire 70 is configured to pull a site marker, such as the site marker 24, into the inner cannula 42 after sterilization and prior to insertion of the inner cannula 42 into the tissue 20. In the embodiment illustrated, the trigger wire 70 is constructed of nitinol, although other suitable materials, such as stainless steels may also be used.

As best seen in FIG. 3, in one embodiment the site marker 24 includes a generally elongated first filament member 80, a second filament member 82, a third filament member 84, fourth filament member 86, a first end connection 88, and a second end connection 90. Each of the filament members 80, 82, 84, 86 extend between the first end connection 88 and the second end connection 90. One of the filament members 80, 82, 84, 86 may be shorter than the others in order to promote the longer filament members to biasingly flex away from the shorter filament member. That is, at least a portion of the filament members 80, 82, 84, 86 are elastically deformed so as to be deformed while maintaining a resiliency that will encourage the filament members to bias toward the configuration illustrated in FIG. 3.

At least one of the filament members 80, 82, 84, 86 are selectively configurable between a first deployed configuration (FIG. 3) and a first retracted configuration (FIG. 6). That is, the site marker 24 is configurable between the first deployed configuration (FIG. 3) and the first retracted configuration (FIG. 6) as at least one of the filament members 80, 82, 84, 86 are deformed, while not all filament members 80, 82, 84, 86 need be deformed to deform the site marker 24 between the first deployed configuration and the first retracted configuration. In the first deployed configuration the site marker 24 can not be interposed within the inner cannula 42. In the first retracted configuration the site marker may be interposed within the inner cannula 42.

The inventors have discovered that some site markers, when sterilized by heat in the retracted configuration (with the site marker completely interposed within an inner cannula, such as the inner cannula 42) may impose a permanent ‘set’ in the site marker. That is, the site marker, while elastically deformed within an inner cannula, may plastically deform due to the application of heat. When a plastically deformed site marker is deployed, the site marker will not fully expand to the deployed configuration, and may migrate from the desired deployment location.

The site marker 24 may be packaged in a sterile enclosure (not shown) in the configuration generally illustrated in FIG. 4. The site marker 24, the inner cannula 42, and at least a portion of the trigger wire 70 may be sterilized in this configuration. This sterilization may take place just prior to deployment of the site marker 24, during manufacture of the system 22, or at any other appropriate time.

In this sterilization configuration of FIG. 4, inner cannula 42 and trigger wire 70 may be connected to a device (not shown) that controls the deployment of the site marker 24. To retract the site marker 24 into the inner cannula 42, the device will restrain the trigger wire 70 in a generally constant position while the inner cannula 42 is moved in the direction of the arrow D, thereby permitting at least a portion of the site marker 24 to remain relatively stationary.

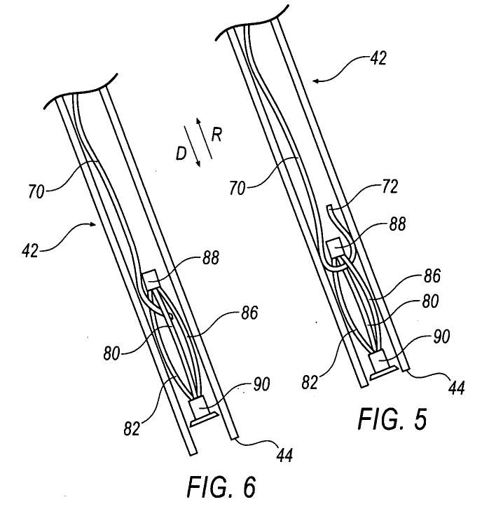

Alternatively, the site marker 24 would be pulled into the inner cannula 42 as the trigger wire 70 is pulled adjacent the proximal wire end (toward the direction of arrow R). The trigger wire 70 is further pulled generally in the direction of the arrow R toward the configuration of FIG. 5. As illustrated in FIG. 5, the site marker 24 begins to disengage from the trigger wire 70 as the trigger wire 70 is further pulled generally in the direction of the arrow R. The trigger wire 70 is then pulled toward the direction of the arrow R, where the site marker 24 disengages from the trigger wire 70 (FIG. 6). The trigger wire 70 may then be removed from the inner cannula 42, may remain generally in the position of FIG. 6, or may be then be pushed toward the distal inner cannula end 44 to force the site marker 24 out of the inner cannula 42 thereby deploying the site marker 24. In lieu of using the trigger wire 70 to deploy the site marker 24, an elongated member, such as a biopsy needle or obturater, or pushrod, may be used to push the site marker 24 in the direction of the arrow D to deploy the site marker 24. Additionally, the inner cannula may be moved in the direction of the arrow R as the site marker 24 and the trigger wire 70 or other component restricts movement of the site marker in the direction of the arrow R, to deploy the site marker 24.

As illustrated, the site marker 24 is deformed when the site marker 24 is interposed within the inner cannula. At least a portion of the site marker 24 is elastically deformed, although the site marker may be partially plastically deformed. That is, not all portions of the filament members 80, 82, 84, 86 may rebound to the sterilization configuration after being released from the inner cannula 42. Additionally, the deployed configuration may be restricted as the site marker 24 contacts portions of the biopsy cavity 30, thereby preventing a full expansion of the site marker 24.

As discussed above, a sterilization configuration is illustrated in FIG. 4. In the sterilization configuration of FIG. 4, the site marker 24 can not be interposed within the inner cannula 42, but must first be deformed to a configuration such as the configurations of either FIG. 5 or 6.

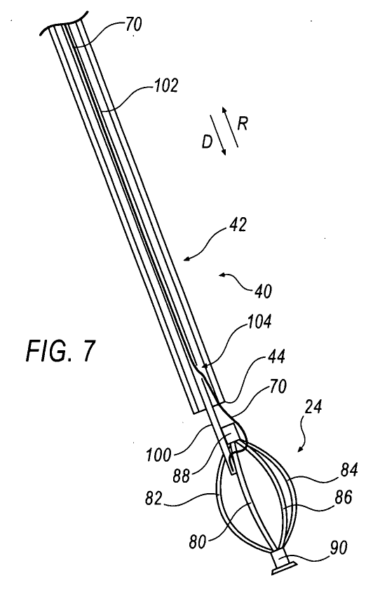

FIG. 7 illustrates an alternative embodiment of the deployment device 40 that includes a push rod 100. The push rod 100 includes a channel 102 formed therein and a channel aperture 104 extending from the channel 102. The trigger wire 70 is inserted through the channel 102 and the channel aperture 104. The push rod 100 may be moved in the direction of the arrow R to retract the site marker 24 within the inner cannula 42, then the trigger wire 70 may be moved in the direction of the arrow R to disengage the trigger wire 70 from the site marker 24, then the push rod 100 moved in the direction of the arrow D to deploy the site marker 24. In the embodiment illustrated, the push rod 100 extends to the distal inner cannula end 44.

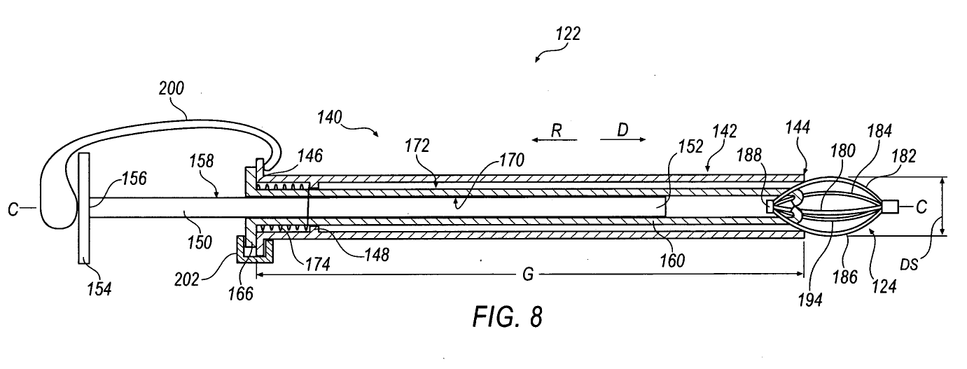

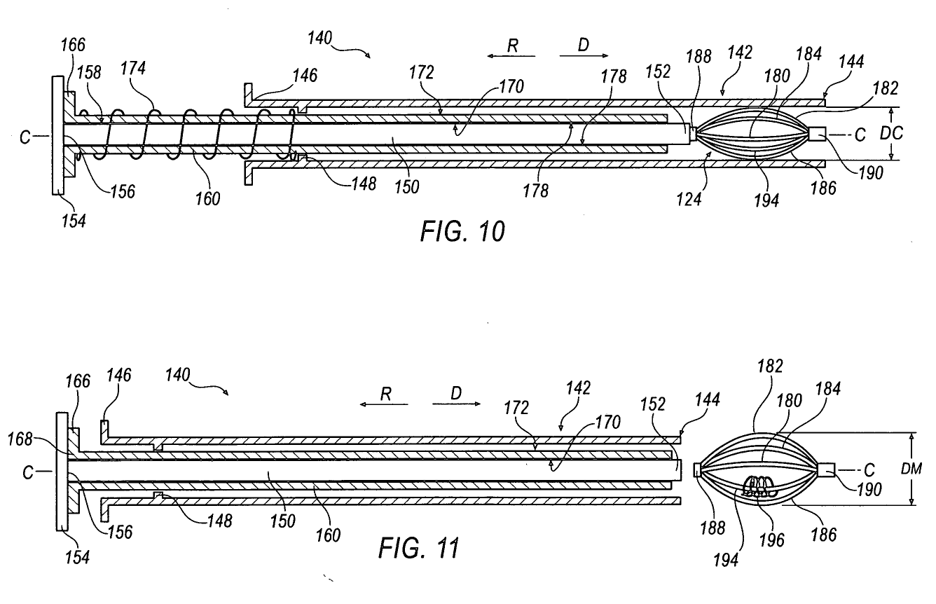

FIGS. 8-12 illustrate another alternative embodiment of the deployment device 40 as a deployment device 140. The deployment device 140 includes an inner cannula 142 having an inner diameter DC (FIG. 10), and a site marker 124. The inner cannula 142 has an axis C-C and extends from a distal inner cannula end 144 to a proximal inner cannula flange 146 to define a length G, and a generally cylindrical inner cannula inner lip 148. As illustrated, the proximal inner cannula flange 146 is formed at an axial end of the inner cannula 142. The deployment device 140 also includes a push rod 150 at least partially interposed within the inner cannula 142 and having a push rod distal end 152, a push rod handle 154 at a push rod proximal end 156, and a generally cylindrical push rod outer surface 158.

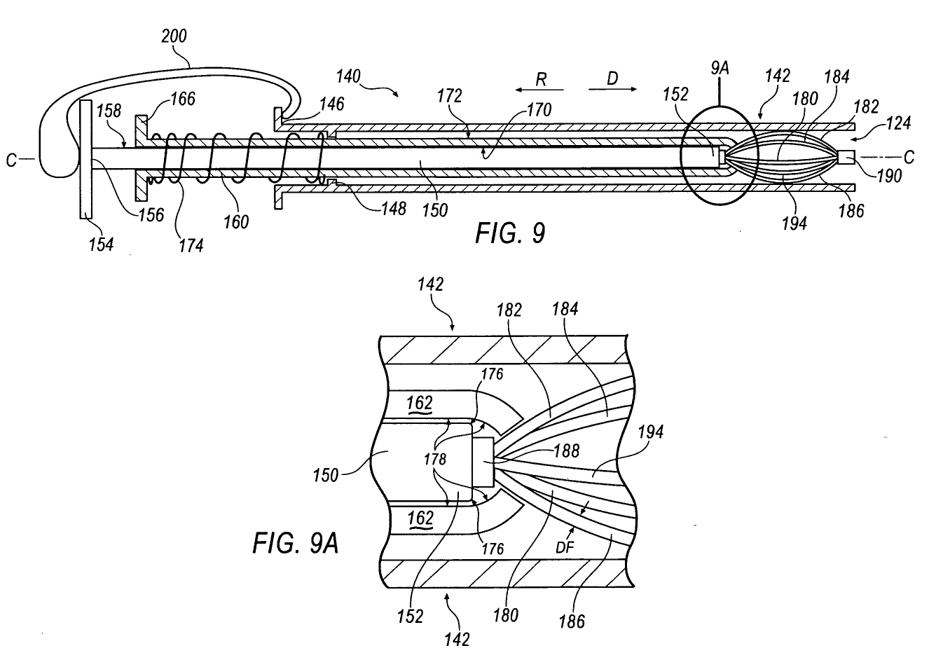

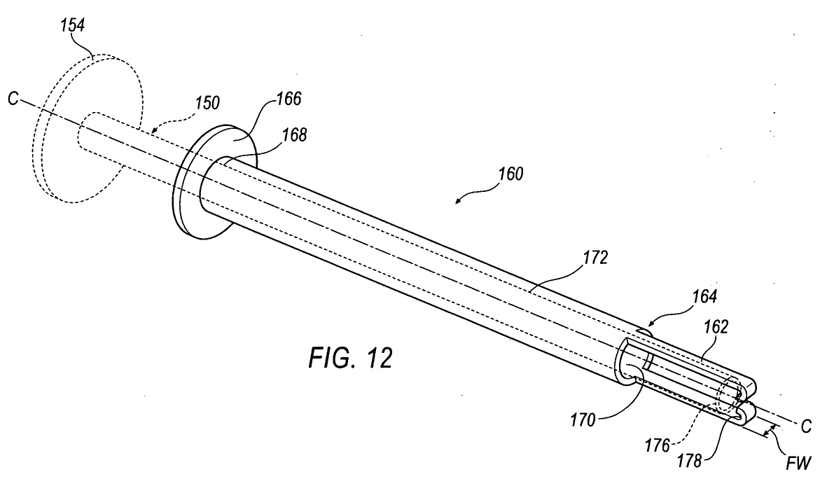

The device 140, further includes a restraining member, or retracting member, 160 having a pair of finger members 162 (as best seen in FIG. 9A) extending from a distal end 164 (FIG. 12), a retracting member flange 166 formed on a proximal end 168, a generally cylindrical inner retracting surface 170, and a generally cylindrical outer retracting surface 172. The retracting member 160 is generally defined by a retracting member thickness TR between the inner retracting surface 170 and the outer retracting surface 172. As best seen in FIG. 12, the retracting member 160 has a generally tubular configuration defined by the inner retracting surface 170 and the outer retracting surface 172, while the fingers 162 have generally rectangular sections when viewed normal to the axis C-C. In the embodiment illustrated, the finger members 162 are constructed of nitinol, although other suitable materials, such as stainless steels may also be used.

The device 140 also includes a biasing member 174 interposed between the inner cannula inner lip 148 and the retracting member flange 166. Thus positioned, the biasing member 174 will urge the retracting member 160 in the direction R relative to the inner cannula 142. In the configuration illustrated in FIG. 8, the retracting member 160 is axially affixed to the inner cannula 142 (discussed below), and in the configuration illustrated in FIG. 10, the retracting member 160 has been un-affixed to the inner cannula 142 to permit the biasing member 174 to urge the retracting member 160 in the direction R relative to the inner cannula 142.

The inner cannula inner lip 148 circumscribes the outer retracting surface 172, while the inner retracting surface 170 circumscribes the push rod outer surface 158. The push rod outer surface 158 is defined by a push rod distal edge 176 (as best seen in FIG. 9A) at the push rod distal end 152. Each finger member 162 has a distal end surface 178 that is selectively guided by the push rod distal edge 176 as best seen in FIG. 9A. That is, the push rod distal edge 176 interferes with the distal end surface 178 of each finger member 162 to deflect (deform) the finger member 162 and disengage the finger members 162 from the site marker 124.

As discussed in greater detail below, the biasing member 174 is adapted to urge the retracting member 160 such that the site marker 124 is urged into the inner cannula 142. The biasing member 174 provides a predetermined force to urge the site marker 124 within the inner cannula 142, thereby preventing a user from providing an undesirable amount of force on the site marker 124.

As best seen in FIG. 8, the site marker 124 includes a generally elongated first filament member 180, a second filament member 182, a third filament member 184, fourth filament member 186, a first end connection 188, and a second end connection 190. In the embodiment illustrated in FIG. 11, the site marker 124 also includes a fifth filament member 194 having a marker element, or permanent marker, 196 (FIG. 1) attached thereto, although the fifth filament member 194 and the permanent marker 196 may be omitted. Each of the filament members 180, 182, 184, 186 are defined by a filament diameter DF (FIG. 9A). The site marker 124, as seen in the configuration of FIG. 11, may be defined by a dimension DM, measured normal to the axis C-C. In the embodiment illustrated, the dimension DM is greater than the inner diameter DC.

Each of the filament members 180, 182, 184, 186, 194 extend between the first end connection 188 and the second end connection 190. In the embodiment illustrated, the filament member 180 is shorter than the filament members 182, 184, 186, 194. Thus configured, the filament member 180 will remain generally straight while the filament members 182, 184, 186, 194 are resiliently curved.

At least one of the filament members 180, 182, 184, 186, 194 is selectively configurable between a first deployed configuration (FIG. 1) and a first retracted configuration (FIGS. 9 and 10). That is, the site marker 124 is configurable between the first deployed configuration (FIG. 1) and the first retracted configuration (FIG. 9) as at least one of the filament members 180, 182, 184, 186, 194 are deformed, while not all filament members 180, 182, 184, 186, 194 need be deformed to deform the site marker 124 between the first deployed configuration and the first retracted configuration. In the first deployed configuration the site marker 124 is in an expanded configuration and generally defined by dimensions, such as the dimension DM, and can not be interposed within the inner cannula 142. In the first retracted configuration the site marker 124 may be interposed within the inner cannula 142 as the site marker 124 is generally defined by the dimension DC when measured normal to the axis C-C. In the sterilization configuration of FIG. 8, the site marker 124 is defined by a sterilization dimension DS when measured normal to the axis C-C.

As best illustrated in FIG. 8, the site marker 124 may be partially interposed within the inner cannula 142 when the site marker is in a sterilization configuration. Thus positioned, the site marker 124 may be sterilized while the filament members 180, 182, 184, 186 are deformed less than the deformation associated with the entire site marker 124 being wholly interposed within the inner cannula 142. The deployment device 140 may be a portion of a system 122 that may be supplied in the sterilization configuration of FIG. 8 and the deployment device 140 may then be sterilized prior to deployment of the site marker 124, or the system may be sterilized in the sterilization configuration of FIG. 8 and supplied and/or stored in this configuration awaiting deployment.

As illustrated in FIG. 8, the device 140 further includes a pushrod retainer 200 and a flange retainer 202. In the embodiment illustrated, the pushrod retainer 200 is removably affixed to the proximal inner cannula flange 146 of the inner cannula 142 and the push rod handle 154 to restrain the pushrod 150 from moving in the direction R relative to the inner cannula 142. The flange retainer 202 axially restrains the retracting member 160 and the inner cannula 142 by releasably coupling to the retracting member flange 166 and the proximal inner cannula flange 146.

In an embodiment of operation, the device 140 may be used as follows. A user removes the device 140 from a sterile package (not shown). The user then detaches the retracting member 160 from the inner cannula 142 by uncoupling the flange retainer 202 from the device 140 to permit the biasing member 174 to expand. As the biasing member 174 expands, the retracting member 160 moves in the direction of arrow R relative to the inner cannula 142 while the pushrod 150 remains generally in a constant axial position relative to the inner cannula 142. As the retracting member 160 moves, the pushrod retainer 200 prevents the pushrod 150 from moving in the direction of the arrow R. As the retracting member 160 moves in the direction of arrow R relative to the inner cannula 142 from the configuration of FIG. 8 to the configuration of FIG. 9, the site marker 124 is retracted within the inner cannula 142 due to the engagement of the fingers 162 with at least a portion of the filament members 180, 182, 184, 186, 194 and the first end connection 188 of the site marker 124. As the retracting member 160 moves in the direction of arrow R relative to the inner cannula 142 from the configuration of FIG. 9 to the configuration of FIG. 10, the finger members 162 are biased into a ‘straightened’ configuration as the distal end surface 178 is selectively guided by the push rod distal edge 176, as best seen in FIG. 9A. Thus retracted, the site marker 124 is positioned within the inner cannula 142 for deployment.

The user then detaches the pushrod retainer 200 from the device 140 and inserts the inner cannula 142 into an introducer, such as the outer cannula 38, to position the device 140 relative to the biopsy cavity 30. The user may then urge the push rod 150 in the direction D to move the site marker 124 out of the inner cannula 124 and into the biopsy cavity 30, or other desired location, as best illustrated in FIG. 11. Alternatively, the push rod 150 may be restrained relatively stationary relative to a desired deployment location, such as the biopsy cavity 30, and the inner cannula 142 may be moved generally in the direction of the arrow R to deploy the site marker 124.

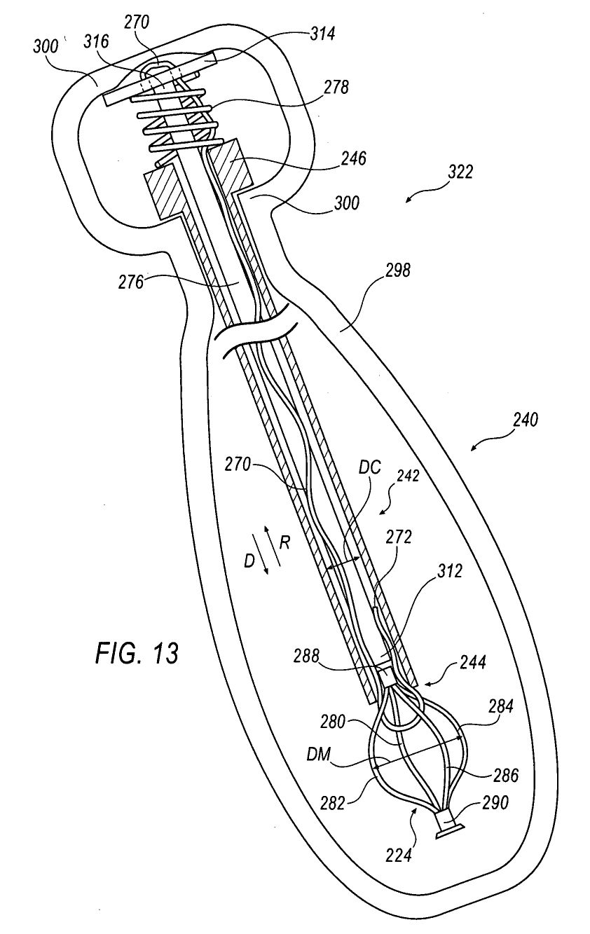

FIGS. 13-15 illustrate yet another alternative embodiment of a deployment device 240. The deployment device 240 includes an inner cannula 242 having an inner diameter of dimension DC and an axis D-D and extending from a distal inner cannula end 244 to a proximal inner cannula end 246.

As discussed in greater detail below, the inner cannula 242 may be interposed within an outer cannula (not shown) that extends from an open proximal end to an open distal end. The outer cannula may be made from a medical grade resin or other MRI compatible material. A depth limiting member, such as a rubber o-ring, may be moveably disposed on outer cannula to limit the insertion depth of outer cannula into the patient's body.

In the embodiment illustrated, the deployment device 240 includes a restraining member, or trigger wire, 270 interposed therein. The trigger wire 270 extends from a distal wire end 272 to a proximal wire end 274. The deployment device 240 also includes a push rod 276 and a biasing member 278. As discussed in greater detail below, the trigger wire 270 is configured to pull a site marker, such as the site marker 224, into the inner cannula 242 after sterilization and prior to insertion of the inner cannula 342 into the tissue 20.

As best seen in FIG. 13, the site marker 224 includes a generally elongated first filament member 280, a second filament member 282, a third filament member 284, fourth filament member 286, a first end connection 288, and a second end connection 290. Each of the filament members 280, 282, 284, 286 extend between the first end connection 288 and the second end connection 290. One of the filament members 280, 282, 284, 286 may be shorter than the others in order to promote the longer filament members to biasingly flex away from the shorter filament member. That is, at least a portion of the filament members 280, 282, 284, 286 are elastically deformed so as to be deformed while maintaining a resiliency that will encourage the filament members to bias toward the configuration of FIG. 13.

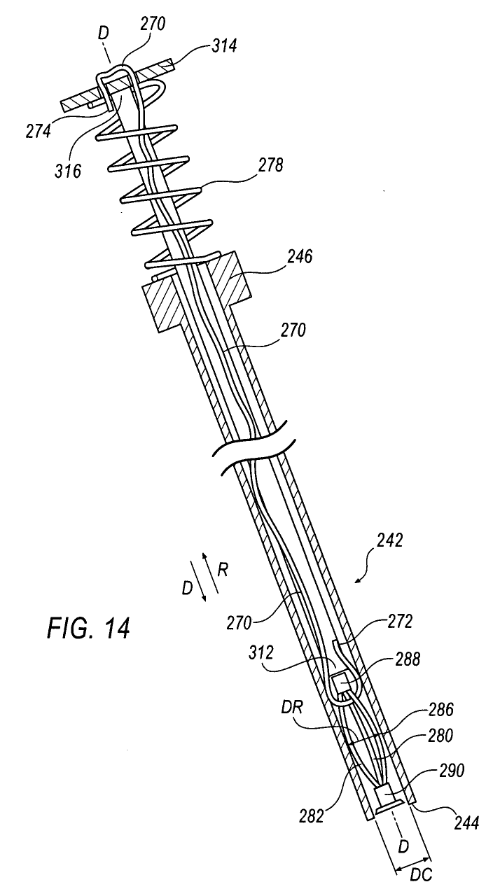

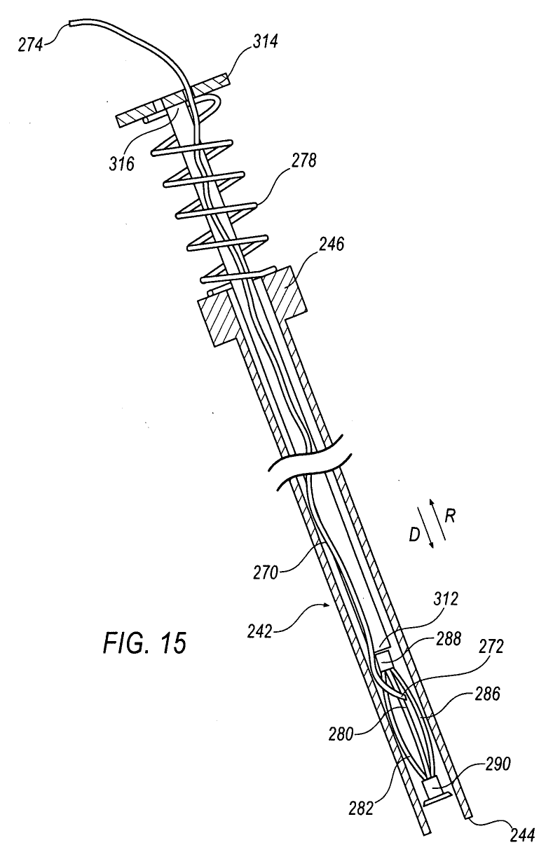

At least one of the filament members 280, 282, 284, 286 is selectively configurable between a first deployed configuration (FIG. 13) and a first retracted configuration (FIG. 15). That is, the site marker 224 is configurable between the first deployed configuration (FIG. 13) and the first retracted configuration (FIG. 15) as at least one of the filament members 280, 282, 284, 286 are deformed, while not all filament members 280, 282, 284, 286 need be deformed to deform the site marker 224 between the first deployed configuration and the first retracted configuration. In the first deployed configuration the site marker 224 is defined by a dimension DM (FIG. 13) and can not be interposed within the inner cannula 242. In the first retracted configuration the site marker 224 is defined by a dimension DR (FIG. 14) and may be interposed within the inner cannula 242.

The push rod 276 is at least partially interposed within the inner cannula 242. The push rod 276 includes a push rod distal end 312, a push rod handle 314 at a push rod proximal end 316, and a generally cylindrical push rod outer surface 318.

The biasing member 278 is interposed between the proximal inner cannula end 246 and the push rod handle 314. Thus positioned, the biasing member 278 will urge the push rod 276 in the direction R relative to the inner cannula 242. In the configuration illustrated in FIG. 13, the biasing member 278 is compressed in a biased configuration. In the configuration illustrated in FIG. 15, the biasing member 278 is uncompressed in an unbiased configuration.

The site marker 224 may be packaged in a sterile enclosure in the configuration generally illustrated in FIG. 13. The site marker 224, the inner cannula 242, and at least a portion of the trigger wire 270 may be sterilized in this configuration.

In this sterilization configuration of FIG. 13, the site marker 224 is positioned such that the site marker 224 will be pulled into the inner cannula 242 as the trigger wire 270 is pulled adjacent the proximal wire end (toward the direction of arrow R). The trigger wire 270 is further pulled generally in the direction of the arrow R toward the configuration of FIG. 15. As illustrated in FIG. 14, the site marker 224 begins to disengage from the trigger wire 270 as the trigger wire 270 is further pulled generally in the direction of the arrow R. The trigger wire 270 is then pulled toward the direction of the arrow R, where the site marker 224 disengages from the trigger wire 270 (FIG. 15). The trigger wire 270 may then be removed from the inner cannula 242, may remain generally in the position of FIG. 15, or may be then be pushed toward the distal inner cannula end 244 to force the site marker 224 out of the inner cannula 242 thereby deploying the site marker 224. In lieu of using the trigger wire 270 to deploy the site marker 224, an elongated member, such as a biopsy needle, may be used to push the site marker 224 in the direction of the arrow D to deploy the site marker 224.

As illustrated, the site marker 224 is deformed when the site marker 224 is interposed within the inner cannula 242. At least a portion of the site marker 224 is elastically deformed, although the site marker 224 may be partially plastically deformed. That is, the filament members 280, 282, 284, 286 may not rebound to the sterilization configuration of FIG. 13 after being deployed from the inner cannula 242. Additionally, the deployed configuration is limited as the site marker 224 interferes with a desired location of insertion, such as the biopsy cavity 30.

As illustrated in FIG. 13, the device 240 is positioned within a packaging 298. The packaging 298 includes a retainer portion 300. In the embodiment illustrated, the retainer portion 300 bindingly retains the inner cannula 242 and the pushrod 276 such that the pushrod 276 is prevented from moving in the direction R relative to the inner cannula 242 and the biasing member 278 is retained toward the biased configuration. Collectively, the site marker 224, the device 240, and the packaging 298 comprise a system 322.

In the embodiment illustrated, FIG. 13 illustrates a sterilization configuration, as discussed in greater detail below. In the sterilization configuration of FIG. 13, the site marker 224 can not be interposed within the inner cannula 242, but must first be deformed to a configuration such as the configurations of either FIG. 14 or 15. Sterilization may take place just prior to deployment of the site marker 224, during assembly of the system 322, or at any other appropriate time.

An embodiment of deploying a site marker using the device 240 is as follows. The device 240 is assembled as generally seen in FIG. 13 and then positioned within the packaging 298 such that the pushrod 276 is axially restrained relative to the inner cannula 242. The device 240 with the packaging 298 may then be sterilized.

After sterilization, the device 240 may be then stored for later use, as the site marker 224 is generally retained in the sterilization configuration of FIG. 13 with the dimension DM greater than the dimension DC.

After removal of the device 240 from the packaging 298, the trigger wire 270 and the push rod 276 are urged by the biasing member 278 generally in the direction of arrow R as the site marker 224 is retracted within the inner cannula toward the position of FIG. 14. Once retracted to about the position of FIG. 15, the deployment device 240 may then deploy the site marker 224.

To deploy the site marker 224, the trigger wire 270 is pulled in the general direction of the arrow R as the push rod is held in a generally unmoved position relative to the inner cannula 242. Once the trigger wire 270 is pulled out of contact with the site marker 224, the trigger wire 270 may be removed from the inner cannula 242 and the inner cannula 242 may be interposed within an outer cannula such as the outer cannula 38 to position the distal inner cannula end 244 adjacent a desired location for marker deployment. The push rod 276 may then be moved generally in the direction of the arrow D until the site marker 224 is no longer interposed within the inner cannula 242, thereby deploying the site marker 224 into the desired location, such as the biopsy cavity 30. After deployment, the device 240 may be removed from the patient.

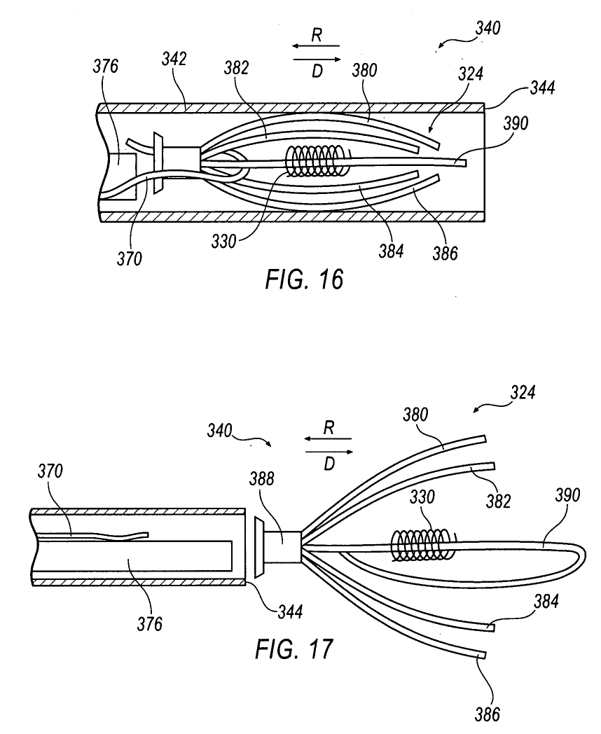

FIGS. 16 and 17 illustrate another embodiment as a deployment device 340. The deployment device 340 includes a site marker 324 and an inner cannula 342. To form the site marker 324, a central filament member 390, which may be a wire or suture material, is attached to a marker end 328. The wire 390 may be constructed from any biocompatible material with suitable echogenic properties such as, but not limited to, titanium, stainless steel, or platinum. Alternatively, the wire 390 may be a bio-absorbable material with a marker element, or permanent marker, 330 coupled to a portion thereof where the permanent marker 330 has suitable echogenic properties. In the embodiment illustrated, the permanent marker 330 has the wire 390 interposed therethrough.

The deployment device 340 also includes a trigger wire 370 and a push rod 376 interposed within the inner cannula 342. The site marker 324 also includes filament members 380, 382, 384, 386 connected to the marker end 328. The central filament member 390 extends from the marker end 328 and may be looped to retain the permanent marker 330.

Similar to the discussion above, the deployment device 340 is sterilized in generally the configuration of FIG. 17, with the site marker 324 partially interposed within the inner cannula and the trigger wire 370 engaged with the site marker 324. The deployment device 340 may then be sterilized in the deployment/sterilization configuration of FIG. 17, with the filament members 380, 382, 384, 386 in a relaxed state with little or no elastic deformation present.

After sterilization, the trigger wire 370 and the push rod 376 are urged generally in the direction of arrow R as the site marker 324 is retracted within the inner cannula toward the position of FIG. 16. Once retracted to about the position of FIG. 16, the deployment device 340 may be inserted into a biopsy cavity and then deploy the site marker 324.

To deploy the site marker 324, the trigger wire 370 is pulled in the general direction of the arrow R as the push rod is held in a generally unmoved position relative to the inner cannula 342. Once the trigger wire 370 is pulled out of contact with the site marker 324, the push rod 376 may be moved generally in the direction of the arrow D until the site marker is no longer interposed within the inner cannula 342, thereby deploying the site marker 324.

As desired, the devices 140, 240 may be positioned within the packaging 298 the pushrod retainer 200, or any suitable device that will axially restrain portions of a device relative to other portions.

In general, the site markers described herein are made, at least in part, from biocompatible materials such as, but not limited to, titanium, stainless steel, and platinum. These materials have appropriate densities for radiographic imaging, appropriate surface characteristics for ultrasonic imaging, and appropriate magnetic characteristics for magnetic resonance imaging. The site markers are preferably made from titanium; however, it is understood that any suitable biocompatible material may be used. Alternatively, the site markers may be made of a bio-absorbable material with a permanent marker attached thereto. In the embodiments illustrated, the filament members have an aspect ratio of at least about 10:1, although other suitable aspect ratios may be used.

After installation in a biopsy cavity, over a predetermined time period such as three weeks to six months, the bio-absorbable materials described herein may be absorbed by the body, such that only permanent marker 196 (if provided), remains within the body at the biopsy cavity 30. Because permanent markers are captured within the tissue 20 prior to absorption thereof by the body, permanent markers are restricted from migrating from within the tissue 20, such as within the biopsy cavity 30. Indeed, movement of a permanent marker is limited to the internal cavity immediately adjacent where a site marker is deployed. This insures that permanent markers remain within an area, such as the biopsy cavity 30 location, after the biopsy cavity 30 has closed to permit follow-up imaging of the biopsy site.

In other embodiments, a site marker may be constructed, at least in part, of a temperature dependent material. These site markers would not fully expand from the retracted configuration into the deployed configuration until heat is applied to the site marker. Deploying the site marker into a biopsy cavity provides a sufficient level of heat generated from the body to encourage the site marker to automatically expand into the second post-deployment configuration after deployment. Such materials include the shape-memory metal nitonol.

Once released from the deployment device and into the biopsy cavity, the site marker automatically springs (due to the elastic deformation) into the deployed configuration having a size and shape defined by the biopsy cavity such that the site marker is easily visible under various imaging modalities.

After installation in a biopsy cavity, such as biopsy cavity 30, over a predetermined time period such as three weeks to six months, the bio-absorbable filament members are absorbed by the body, such that only a permanent marker remains within the body within the biopsy cavity location, and is visible under one or more modalities such as X-ray, magnetic resonance imaging (MRI), or ultrasound imaging. Filament member may be absorbed by tissue ingrowth, leaving only a permanent marker, which may be retained in place by the tissue ingrowth.

In the embodiments illustrated, the permanent markers may be constructed of a material that is not absorbed by the body. Alternatively, the permanent markers may be a semi-permanent marker that bio-absorbs slower than the filament member. Because the movement of the permanent markers is restricted by the filament members prior to absorption thereof by the body, the permanent markers are restricted from migrating from within biopsy cavity. This insures that the permanent markers remain within the biopsy cavity to permit follow-up imaging of the biopsy site. The permanent marker may also be contained within the filament members to ‘free float’ while not being attached to any portion of the site marker. Further, the first end connection 88, 188, 288, 388, and/or the second end connection 90, 190, 290 may be constructed of a material that has permanent marker qualities.

The filament member may have an aspect ratio of greater than about 10:1, or other suitable dimensions, to prevent migration of the site marker from a biopsy site. Further, a site marker end cap may be ‘permanent’ materials having suitable echogenic properties such as, but not limited to, titanium, stainless steel, or platinum.

Since a site marker, such as the site marker 24, 124, 224, 324 may be deployed with the aid of a MRI, the user will visually detect when the site marker has been deployed and may confirm that the site marker has been successfully deployed in the desired location.

Although the steps of the method of deploying the site markers described herein are listed in a preferred order, the steps may be performed in differing orders or combined such that one operation may perform multiple steps. Furthermore, a step or steps may be initiated before another step or steps are completed, or a step or steps may be initiated and completed after initiation and before completion of (during the performance of) other steps.

Among other features, the medical system of the present invention localizes the target biopsy site in a manner that allows confirmation of the target biopsy site under MRI or other visualization modality, and allows increased accuracy of positioning of a biopsy device to ensure the cutting element of the biopsy device can be accurately placed at the target biopsy site. The medical system also prevents migration of site markers by reducing any predeployment plastic deformation of the site markers, thereby permitting the site markers to expand and engage the inside surfaces of a biopsy cavity with a greater force.

While the embodiments of site markers 24, 124, 224, 324 are described as having four filament members, it is understood that one or more filament members may be adequate to retain the marker in the desired biopsy cavity or other location. In addition, while the present invention has been particularly shown and described with reference to the foregoing preferred embodiments, it should be understood by those skilled in the art that various alternatives to the embodiments of the invention described herein may be employed in practicing the invention without departing from the spirit and scope of the invention as defined in the following claims. It is intended that the following claims define the scope of the invention embodiments within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A site marker deployment system comprising:
a deployment device having an inner cannula;
a site marker that may be selectively interposed within the inner cannula, wherein the site marker includes at least one generally elongated filament member, the filament member is selectively configurable between a first configuration, where the site marker can not be interposed within the inner cannula, and a second configuration, where the site marker may be interposed within the inner cannula, a portion of the site marker being elastically deformed when the site marker is interposed within the inner cannula; and
a restraining member selectively interposed within the inner cannula and selectively engaging the site marker such that movement of the restraining member relative to the inner cannula may urge the site marker at least partially into the inner cannula.

2. The system of claim 1, further comprising a push rod selectively interposed within the inner cannula, wherein the push rod will selectively guide the site marker out of the inner cannula.

3. The system of claim 2, wherein the push rod will disengage the restraining member from the site marker as the restraining member is moved relative to the push rod.

4. The system of claim 2, wherein the push rod is defined by a channel and the restraining member is selectively at least partially interposed within the channel.

5. The system of claim 1, further comprising an outer cannula, wherein the outer cannula is configured to introduce at least one of the inner cannula, a biopsy device, a site marker, an anesthesia, a fluid, a tamponade, and a hemostatic agent.

6. The system of claim 1, wherein the site marker further includes a permanent marker element, wherein the marker element has an aperture that receives the filament member therethrough.

7. The system of claim 1, wherein the filament member has an aspect ratio of greater than about 10:1.

8. The system of claim 1, wherein the restraining member is constructed of a stainless steel or a nitonol.

9. The system of claim 1, wherein the restraining member is an elongated trigger wire.

10. The system of claim 9, further comprising a push rod selectively interposed within the inner cannula, the push rod including a proximal end and a distal end, wherein the trigger wire extends at least partially through the inner cannula, past the push rod distal end, and loops back within the inner cannula for urging the site marker completely within the inner cannula.

11. The system of claim 9, wherein the trigger wire may be disengaged from the site marker such that movement of the trigger wire away from the site marker will not move the site marker.

12. The system of claim 1, wherein the restraining member includes at least one finger member.

13. The system of claim 12, further comprising a push rod selectively interposed within the inner cannula, wherein the at least one finger member has a distal end surface that selectively interferes with a surface of the pushrod to deflect at least a portion of the finger member for disengaging the restraining member from the site marker.

14. The system of claim 1, further comprising a biasing member for urging the restraining member in a first direction such that the site marker is urged within the inner cannula.

15. A site marker deployment system comprising:
a site marker, wherein the site marker includes a generally elongated first filament member, the first filament member is selectively configurable between a first deployed configuration, where the site marker can not be interposed within the inner cannula, and a retracted configuration, wherein the site marker is selectively interposed within the inner cannula, and wherein a portion of the site marker is elastically deformed when the site marker is interposed within the inner cannula;
a deployment device having an inner cannula; wherein the inner cannula is defined by an axis, the inner cannula for may selectively retain the site marker, wherein the site marker is selectively configurable in a sterilization configuration where at least a portion of the site marker is not interposed within the inner cannula; and
a biasing member selectively configurable between a biased configuration and an unbiased configuration, for urging the site marker into the inner cannula as the biasing member moves between the biased configuration and the unbiased configuration.

16. The system of claim 15, further comprising a push rod at least partially interposed within the inner cannula, wherein the site marker will selectively move relative to the inner cannula as the push rod moves axially relative to the inner cannula.

17. The system of claim 16, further comprising a restraining member selectively interposed within the inner cannula and selectively engaging the site marker such that a force exerted on the restraining member by the biasing member may urge the site marker at least partially into the inner cannula.

18. The system of claim 17, further comprising a push rod selectively interposed within the inner cannula, wherein the restraining member is a finger member having a distal end surface that interferes with a surface of a pushrod as the restraining member is moved relative to the push rod to deflect at least a portion of the finger member to disengage the finger member from the site marker.

19. The system of claim 15, wherein site marker also includes a permanent marker coupled to the first filament member.

20. The system of claim 15, wherein the first filament member has an aspect ratio of greater than about 10:1.

21. The system of claim 15, wherein the site marker further includes a second filament member and a third filament member.

22. The system of claim 21, wherein the restraining member is interposed between the first filament member and the second filament member.

23. The system of claim 21, wherein the site marker is defined, at least in part, by an end connection that connects the first filament member with the second filament member and the third filament member

24. The system of claim 23, wherein the site marker further includes a second end connection connected to at least one of the first filament member, the second filament member, and the third filament member.

25. The system of claim 15, wherein at least a portion of the deployment device is positioned in an interference fit within a retainer such that the biasing member is biased toward the biased configuration.

26. A method of deploying a site marker comprising:
providing a site marker deployment device having an inner cannula and a site marker, wherein the site marker includes at least one generally elongated filament member that is selectively configurable between a first deployed configuration where the site marker can not be interposed within the inner cannula, and a retracted configuration where the site marker may be interposed within the inner cannula, a portion of the site marker being elastically deformed when the site marker is interposed within the inner cannula, and wherein the site marker is selectively configurable in a sterilization configuration where at least a portion of the site marker is not disposed within the inner cannula;
sterilizing the site marker in the sterilization configuration; and
engaging at least a portion of the site marker with a portion of the deployment device, wherein another portion of the deployment device may be manipulated such that the portion of the deployment device urges the site marker into the inner cannula as the site marker is reconfigured from the sterilization configuration to the retracted configuration.

27. The method of claim 26, further comprising interposing the site marker within the inner cannula.

28. The method of claim 26, wherein providing a site marker deployment device includes providing the site marker where at least at least a portion of the site marker is constructed of a bio-absorbable material.

29. The method of claim 26, further comprising deploying the site marker into a desired location, wherein the site marker expands to a deployed configuration.

30. The method of claim 26, wherein interposing the site marker within the inner cannula includes moving a trigger wire.

31. The method of claim 26, wherein interposing the site marker within the inner cannula includes moving a restraining member having a curved finger member.

32. The method of claim 26, wherein interposing the site marker within the inner cannula includes permitting a biasing member to selectively urge the site marker into the inner cannula

33. The method of claim 26, wherein interposing the site marker within the inner cannula is completed after sterilizing the site marker in the sterilization configuration.

34. The method of claim 26, further comprising disengaging the site marker from a restraining member.

35. The method of claim 34, wherein disengaging the site marker from a restraining member includes guiding a distal end surface of the restraining member by a surface of a pushrod to deflect at least a portion of the restraining member.

36. The method of claim 26, further comprising interposing at least a portion of the deployment device into a retainer such the retainer biases a restraining member toward a biased configuration.
